(19) United States
(12) Patent Application Publication
(10) Pub. No.: US 2006/0154898 A1
(43) Pub. Date: Jul. 13, 2006

(54) Diphosphonate solutions

(76) Inventors: Gregory Paul Handreck, Fitzroy (AU); Wei Zhou, Ormond (AU); Russell John Tait, Deepdene (AU)

Correspondence Address: REED SMITH LLP, P.O. BOX 488, PITTSBURGH, PA, 15230-0488, US

(21) Appl. No.: 11/194,106

(22) Filed: Jul. 28, 2005

Related U.S. Application Data
(63) Continuation of application No. 10/380,645, filed on Dec. 19, 2003.

(30) Foreign Application Priority Data
Sep. 18, 2000 (AU) PR 0189
May 10, 2001 (AU) PR 4855

(51) Int. Cl. A61K 31/675; A61K 31/66; B65D 85/42

(52) U.S. Cl. 514/89; 514/102; 206/530

(57) ABSTRACT

A pharmaceutical product containing pamidronate and other diphosphonate solutions in an appropriate container, a pH of between 5 and 8 and without organic acid buffer or polyethylen glycol. The container may be treated glass or made of other appropriate material. Coated elastomeric stoppers are also included. A method of producing a pharmaceutical product comprising steps of making a suspension of pamadronic acid, adding sodium hydroxide, to form a solution adjusting the pH to between 5 and 8 and transferring the solution to a container.

### FIELD OF THE INVENTION

This invention relates to stable injectable solutions containing diphosphonates.

### BACKGROUND

Various diphosphonic acids can be used as therapeutic active agents for the treatment of hypercalcaemia and in medication for the treatment of diseases such as osteoporosis and tumor osteolysis. In a prepared solution, the active agent will be present as anions and is generally called diphosphonate, bisphosphonate or biphosphonate. An injection solution of diphosphonate can be prepared from the diphosphonic acid or one of its salts. A convenient method for administering these active agents is by intravenous infusion of prepared solutions into the bloodstream of a patient to be treated.

One diphosphonic acid, pamidronic acid is currently available in the form of a lyophilised product to be reconstituted prior to use. This reconstitution step involves not only both time and effort, but also introduces the possibility of adverse consequences through, for example improper reconstitution and mixing of the powder and contamination prior to administration. Previous efforts to formulate pamidronate solutions have suffered from stability problems with the solutions showing turbidity and loss of active product over time.

Glass has long been the material of choice for containers for pharmaceutical products. However, it has been found that diphosphonate solutions left in glass for extended periods display unacceptable levels of turbidity despite the good solubility and chemical stability of diphosphonates generally.

It is known that the level of turbidity of diphosphonate solutions in glass is affected by the pH of the solution, and that the level of turbidity decreases with increased acidity.

An approach to minimise the problem of reaction between the active substances and glass leachates is the use of excipients such as polyethylene glycols or acid buffers such as organic acids. Whilst the use of such excipients may assist, it is generally preferable to minimise the number of additional constituents of any injectable product solution.

### SUMMARY OF THE INVENTION

Surprisingly, the inventors have found that it is possible to formulate stable diphosphonate solutions such as, in particular, pamidronate, which are neither highly acidic nor which involve the use of buffer systems. The inventors have found that solutions of diphosphonates of relatively neutral pH values do exhibit satisfactory stability provided appropriate containers are used.

This invention provides a stable and preprepared injectable solution of diphosphonate ready to be diluted by a practitioner administering the product to the patient. This enables the product to be provided in a consistent quality and avoids the need for the practitioner to reconstitute the active agent at the time of administration.

According to one aspect the present invention provides a pharmaceutical product comprising a container containing a diphosphonate in solution, wherein the solution:
(a) has a pH of between 5 and 8;
(b) is free of organic acid buffer and polyethylene glycol;
   and wherein the container consists of at least one component manufactured from glass having at least a surface in contact with the solution, at least one said surface having been pre-treated to protect against the leaching of impurities from the glass by the solution.

According to a further aspect, the present invention provides a pharmaceutical product comprising a container containing a diphosphonate in solution, wherein the solution:
(a) has a pH of approximately 6.5; and
(b) is free of organic acid buffer and polyethylene glycol
   and wherein the container consists of at least one component manufactured from glass having at least a surface in contact with the solution, at least one said surface having been pre-treated so as to protect against the leaching of impurities from the glass by the solution.

According to a further aspect, the present invention provides a pharmaceutical product comprising a container containing a diphosphonate in solution, wherein the solution:
(a) has a pH of between 5 and 8;
(b) is free of organic acid buffer and polyethylene glycol; and
   wherein the container consists of at least one component manufactured from a non-glass material.

According to a further aspect the present invention provides a method of preparing a pharmaceutical product, said method comprising the steps of:
(a) preparing a suspension of pamodronic acid in water;
(b) adding sodium hydroxide solution to the suspension to obtain a second solution;
(c) adjusting the pH of the second solution to between 5 and 8; and
(d) transferring the second solution to a container.

### DESCRIPTION

In order to obtain adequate long-term stability, appropriate containers must be used for the solution of diphosphonate. Appropriate containers for this product include ampoules, vials, bottles, ready to use syringes and Shell Glass Vials.

It is believed that the principal cause of turbidity where glass containers have been used in the past is the leaching out from the glass of aluminium and/or other cations such as magnesium or calcium, depending upon the glass composition.

Where glass containers are used it is necessary to pre-treat the contact surface of the glass with an appropriate method to protect against the leaching of impurities from the glass by the solution. Preferably all potential contact surfaces will be appropriately treated. In this way, the extent to which impurities leach from the glass over time is reduced. A preferred method of pre-treatment is a siliconization process using a one percent silicone solution to wash the vials, followed by double draining and heating at 310° C. for thirty minutes. Vials pretreated in this manner are available from the French vial manufacturer Saint-Gobain Desjonqueres (SGD).

Other vial pretreatment techniques include the use of a high purity SiO₂ barrier formed on the inside vial surface by a plasma-deposition process. The process involves microwave energy being applied to a silicon containing precursor in the presence of oxygen. A plasma forms and a SiO₂ layer is formed on the glass surface from the gas phase. Vials pretreated in this manner are available from Schoft.

In addition to treating the surface of the glass, it is also recommended to use containers which are made from glass having a low aluminium content. Glass typically used for pharmaceutical vials has in the order of 5 percent aluminium oxide. In order to reduce the problem of aluminium ion leaching, glass with lower aluminium content is recommended.

Where the solution is stored in a stoppered vial, the stopper provides a potential source of contamination. Typical elastomeric stoppers are potentially a source of metal ions eg calcium, zinc and magnesium ions which can react with the diphosphonate to form insoluble matter. In order to reduce the possibility of contamination, stoppers with low levels of these ions and other potential contaminants are to be used, preferably coated to form an inert barrier. An example of an appropriate stopper is the Daikyo D777-1 stopper. Daikyo D777-3 stoppers may also be used. Preferably the stopper has a low calcium, magnesium and ash content and is at least coated on the contact surface (being the surface of the stopper which when placed in a vial is exposed to the contents of the vial) with a fluorinated resin such as tetrafluoroethylene polymer, trifluorochloroethylene polymer, tetrafluoroethylene-hexafluoropropylene copolymer, fluorovinylidene polymer, vinylidene fluoride polymer, vinyl fluoride polymer, tetrafluoroethylene-ethylene copolymer, ethylene-tetrafluoroethylene copolymer, or perfluoroalkoxy polymer. It is more preferred that the stopper is coated with a fluorinated resin selected from a group consisting of tetrafluoroethylene polymer, trifluorochloroethylene polymer, tetrafluoroethylene-hexafluoropropylene copolymer, vinylidene fluoride polymer, vinyl fluoride polymer, and tetrafluoroethylene-ethylene copolymer. For example, the stopper can be a FluroTec® stopper manufactured by Daikyo and distributed by West Pharmaceuticals Services.

Containers, such as vials, may be constructed from any suitable other materials in addition to glass, such as polyethylene, polypropylene and polymethylpentene. For example, the vial could be constructed from Crystal Zenith® resin as manufactured by West Pharmaceuticals Services.

This invention is generally applicable to all diphosphonates. Specifically, this includes solutions of pamidronate, zolindronate, chlodronate, etidronate, alendronate and tiludronate. These can be prepared from their respective diphosphonic acid form or from a therapeutically acceptable salt form. The acids of the above diphosphonates are: pamidronic acid [(3-amino-1-hydroxypropylidene) diphosphonic acid], zoledronic acid [(1)-hydroxy-2-(1H-imidazol-1-yl)ethylidene) diphosphonic acid]; chlodronic acid [dichloromethylene disphosphonic acid]; etidronic acid [(1-hydroxyethylidene) diphosphonic acid]; alendronic acid [(4-amino-1-hydroxy-butylidene diphosphonic acid]; and tiludronic acid [[((p-chloro-phenyl)thio)methylene] diphosphonic acid] respectively. This invention is particularly applicable to pamidronate and zolendronate.

It is preferred to have a product within the biological pH range i.e. of between about 5 and 8, to reduce the incidence of potential adverse reactions relating to acidic or alkaline solutions. Surprisingly it has been found that a stable solution can be produced having a pH of 5-8. A pH level of approximately 6.5 is preferred. At pH levels below about 5 there is a risk of producing venous type irritations and other unwanted side effects. pH levels above about 8 give rise to generally unacceptable levels of turbidity.

Solutions of diphosphonates will generally have a pH above that desired. For example, a solution of one percent pamidronate disodium salt in distilled water has a pH of approximately 8.3. The pH is adjusted with a suitable acid or alkali. Suitable acids include any acid such as hydrochloric or phosphoric acid. Phosphoric acid is preferred. Suitable alkalis include sodium hydroxide.

As the person skilled in the art will appreciate, other standard components, such as sugar alcohols and sodium chloride and water may be included in the solution, as required. Mannitol is the preferred sugar alcohol.

Pamidronate solutions are preferably prepared by slowly adding sodium hydroxide solution to a suspension of pamidronic acid in water in a 2:1 molar ratio of sodium hydroxide to pamidronic acid, adding mannitol if desired, mixing by stirring until both pamidronic acid and mannitol (if appropriate) are completely dissolved and adjusting the pH with phosphoric acid and if necessary sodium hydroxide solution. Preferably the preparation of the solutions is carried out under nitrogen. Other diphosphonate solutions can be prepared in analogous fashion.

### EXAMPLE 1

Preparation of Pamidronate Solution.

To a mixing vessel approximately 10% of the required amount of Water for Injection is added and then bubbled with nitrogen gas for at least 15 minutes. The sodium hydroxide, in an amount to give a 2:1 molar ratio to pamidronic acid is then added with stirring to dissolve and the solution cooled to less than 30° C.

A different closed mixing vessel is flushed with nitrogen gas for at least 15 minutes. Approximately 70% of the Water for Injection is added to the closed mixing vessel through a port and the mixing bubbled with nitrogen gas for at least 15 minutes. Pamidronic acid is then added to the mixing vessel with stirring and mixed for 5 minutes giving a suspension. The sodium hydroxide solution is then added over a 5 minute period with stirring to give a clear solution. Mannitol is then added to the solution with stirring for at least 5 minutes until dissolved. The pH is then checked and adjusted to a range of between 5 and 8 preferably, between 6.3 and 6.7 by addition of 1.0N phosphoric acid at the rate of approximately 12.1 g/L (calculated on total batch size) and if necessary 1.0N sodium hydroxide, whilst keeping the temperature between 35° C. and 45° C. The volume is adjusted to the required level with Water for Injection and the solution cooled to below 30° C. The pH is then rechecked and adjusted if necessary to between 6.3 and 6.7, with 1.0N phosphoric acid or 1.0N sodium hydroxide if and as necessary.

### EXAMPLE 2

In this example the product solution was composed of the following:

| | |
|---|---|
| pamidronic acid | 2.53 mg |
| mannitol | 47.0 mg |
| sodium hydroxide | 0.43 mg |
| pH | qs to 6.3-6.7 using 1.0N sodium hydroxide or 1.0N |
| | phosphoric acid |
| Water for Injection | qs to 1.0 mL |

The formulated solution was filled into 10 mL siliconised, low aluminium, Type I glass vials, supplied by SGD. Each vial was enclosed by a 20 mm, S10-F451, D777-1, B2-40, FluroTec® stopper supplied by West Pharmaceuticals Services.

Table 1 shows the test results measured over a 24 month period while being stored inverted at 25° C., relative humidity (RH) 60%.

TABLE 1

| | Initial (0 | | | | |
|---|---|---|---|---|---|
| | months) | 6 months | 12 months | 18 months | 24months |
| Appearance | N | N | N | N | N |
| Potency | 98.7% | 99.9% | 99.8% | 100.1% | 99.1 |
| pH | 6.4 | 6.2 | 6.3 | 6.4 | 6.5 |
| Metal ions | | | | | |
| silicon ppm | 0.6 | | 2.9 | | 2.9 |
| calcium ppm | 0.09 | | 0.05 | | 0.07 |
| aluminium | 0.05 | | 0.12 | | 0.11 |
| ppm | | | | | |

N - Clear colourless solution, free from visible particles.

### EXAMPLE 3

In this example the product solution was composed of the following:

| | |
|---|---|
| pamidronic acid | 7.58 mg |
| mannitol | 37.5 mg |
| sodium hydroxide | 1.29 mg |
| pH | qs to 6.3-6.7 using 1.0N sodium hydroxide or 1.0N |
| | phosphoric acid |
| Water For Injection | qs to 1.0 mL |

The formulated solution was filled into 10 mL siliconised, low aluminium, Type I glass vials, supplied by SGD. Each vial was enclosed by a 20 mm, SIO-F451, D777-1, B2-40, FluroTec® stopper supplied by West Pharmaceuticals Services.

Table 2 shows the test results measured over a 24 month period while being stored inverted at 25° C., relative humidity (RH) 60%.

TABLE 2

| | Initial (0 | | | | |
|---|---|---|---|---|---|
| | months) | 6 months | 12 months | 18 months | 24 months |
| Appearance | N | N | N | N | N |
| Potency | 100.2% | 101.9% | 100.4% | 102.3% | 101.1 |
| pH | 6.4 | 6.3 | 6.4 | 6.3 | 6.5 |
| Metal ions | | | | | |
| silicon ppm | 0.6 | | 6.2 | | 12.9 |
| calcium ppm | <0.04 | | 0.1 | | 0.24 |
| aluminium | 0.06 | | 0.25 | | 0.56 |
| ppm | | | | | |

N - Clear colourless solution, free from visible particles.

### EXAMPLE 4

In this example the product solution was composed of the following:

| | |
|---|---|
| pamidronic acid | 2.53 mg |
| mannitol | 47.0 mg |
| sodium hydroxide | 0.86 mg |
| pH | qs to 6.3-6.7 using 1.0N sodium hydroxide or 1.0N |
| | phosphoric acid. |
| Water For Injection | qs to 1.0 mL |

The formulated solution was filled into 10 mL siliconised, low aluminium, Type I glass vials, supplied by SGD. Each vial was enclosed by a 20 mm, S10-F451, D777-1, B2-40, FluroTec® stopper supplied by West Pharmaceuticals Services.

Table 3 shows the test results measured over a 21 month period while being stored inverted at 25° C., relative humidity (RH) 60%.

TABLE 3

| | Initial (0 | | | | |
|---|---|---|---|---|---|
| | months) | 6 months | 12 months | 18 months | 21 months |
| Appearance | N | N | N | N | N |
| Potency | 103.6% | 103.5% | 104.0% | 104.0 | 104.5 |
| pH | 6.5 | 6.4 | 6.5 | 6.6 | 6.5 |
| Metal ions | | | | | |
| silicon ppm | 0.31 | 0.2 | 0.47 | — | — |
| calcium ppm | 0.06 | <0.04 | <0.04 | — | — |
| aluminium | 0.17 | <0.04 | <0.04 | — | — |
| ppm | | | | | |

N - Clear colourless solution, free from visible particles.

### EXAMPLE 5

In this example the product solution was composed of the following:

| | |
|---|---|
| pamidronic acid | 7.58 mg |
| mannitol | 37.5 mg |
| sodium hydroxide | 2.58 mg |
| pH | qs to 6.3-6.7 using 1.0N sodium hydroxide or 1.0N |
| | phosphoric acid. |
| Water For Injection | qs to 1.0 mL |

The formulated solution was filled, into 10 mL siliconised, low aluminium, Type I glass vials, supplied by SGD. Each vial was enclosed by a 20 mm, S10-F451, D777-1, B2-40, FluroTec® stopper supplied by West Pharmaceuticals Services.

Table 4 shows the test results measured over a 21 month period while being stored inverted at 25° C., relative humidity (RH) 60%.

TABLE 4

| | Initial (0 | | | | |
|---|---|---|---|---|---|
| | months) | 6 months | 12 months | 18 months | 21 months |
| Appearance | N | N | N | N | N |
| Potency | 98.9% | 99.2% | 100.0% | 99.1 | 99.4 |
| pH | 6.5 | 6.4 | 6.5 | 6.6 | 6.5 |
| Metal ions | | | | | |
| silicon ppm | 0.29 | 0.3 | 0.65 | — | — |
| calcium ppm | 0.18 | 0.10 | 0.13 | — | — |
| aluminium | 0.12 | <0.04 | 0.07 | — | — |
| ppm | | | | | |

N - Clear colourless solution, free from visible particles.

In each of the examples 2 to 5 above, the solution was prepared by the process set out in Example 1.

It is understood that various modifications, alternatives and/or additions may be made to the product specifically described herein without departing from the spirit and ambit of the invention.

Throughout the description and claims of this specification the word “comprise” and variations of that word such as “comprises” and “comprising” are not intended to exclude other additives, components, integers or steps.

1-27. (canceled)

28. An injectable, sterile, ready to use, pyrogen-free pamidronate solution comprising a physiologically accepted alkaline salt of pamidronate which is water soluble and a physiologically acceptable aqueous solvent, wherein the solution is provided in a sealed non-reactive plastic container.

29. The solution according to claim 28, wherein the physiologically acceptable alkaline salt is a sodium salt.

30. The solution according to claim 28, wherein the physiologically acceptable alkaline salt is a disodium salt.

31. The solution according to claim 30, wherein pamidronate is present in a concentration of between about 2.58 mg/mL and about 7.58 mg/mL.

32. The solution according to claim 31, wherein the concentration is about 2.58 mg/mL.

33. The solution according to claim 31, wherein the concentration is about 7.58 mg/mL.

34. The solution according to claim 31, wherein the sealed container is made of a material selected from the group consisting of polypropylene, polyolefin, and polyethylene.

35. The solution according to claim 34, wherein the sealed container is polypropylene.

36. The solution according to claim 34, wherein the sealed container is closed with a non-reactive stopper.

37. The solution according to claim 36, wherein the stopper is selected from the group consisting of a Teflon-coated stopper and a Teflon-faced stopper.

38. The solution according to claim 34, further comprising mannitol.

39. The solution according to claim 35, wherein the solution has a pH of approximately 6.5.

40. The solution according to claim 39, wherein the aqueous solvent is water.

41. A process for producing a sterile, injectable, pyrogen-free, ready-to-use pamidronate solution comprising adding pamidronic acid to an aqueous solvent to form a pamidronate solution, wherein the aqueous solvent contains sodium hydroxide, filling the pamidronate solution into non-reactive plastic containers, and sealing the containers.

42. The process according to claim 41, wherein sodium hydroxide is added to the aqueous solvent prior to the addition of pamidronic acid.

43. The process according to claim 42, wherein the molar ratio of sodium hydroxide to pamidronic acid is 2:1.

44. The process according to claim 43, wherein the sealed plastic container is a vial.

45. The process according to claim 44, wherein the sealed container additionally comprises a non-reactive stopper.

46. The process according to claim 43, further comprising adjusting the pH of the pamidronate solution to about 5 to about 8.

47. The process according to claim 46, wherein the pH of the pamidronate solution is adjusted to approximately 6.5.

48. A process for manufacturing an aqueous solution of pamidronate, comprising adding pamidronic acid to an aqueous sodium hydroxide solution to form the disodium salt of pamidronate.

49. The process according to claim 48, wherein the molar ratio of sodium hydroxide to pamidronic acid is 2:1.

50. The process according to claim 49, wherein the required amount of sodium hydroxide is present in the aqueous sodium hydroxide solution prior to the addition of pamidronic acid.

51. An injectable pamidronate solution comprising a salt of pamidronate and water, wherein the solution is filled into a stoppered inert container.

52. The solution according to claim 51, wherein the salt is a sodium salt.

53. The solution according to claim 51, wherein the salt is a disodium salt.

54. The solution according to claim 53, wherein pamidronate is present in a concentration of between about 2.53 and about 7.58 mg/mL.

55. The solution according to claim 54, wherein the concentration is about 2.53 mg/mL.

56. The solution according to claim 54, wherein the concentration is about 7.58 mg/mL.

57. The solution according to claim 54, wherein the stoppered container is constructed of a material selected from the group consisting of polypropylene, polyolefin, and polyethylene.

58. The solution according to claim 57, wherein the stoppered container is constructed of polypropylene.

59. The solution according to claim 57, wherein the stoppered container is enclosed with an inert stopper.

60. The solution according to claim 59, wherein the stopper is coated with tetrafluoroethylene polymer.

61. The solution according to claim 57, further comprising mannitol.

62. The solution according to claim 58, wherein the solution has a pH of approximately 6.5.

63. A process for producing an injectable pamidronate solution comprising adding sodium hydroxide solution to pamidronic acid to form a pamidronate solution, filling the pamidronate solution into inert containers, and stoppering the containers.

64. The process according to claim 63, wherein the molar ratio of sodium hydroxide to pamidronic acid is 2:1.

65. The process according to claim 64, wherein the stoppered container is a vial.

66. The process according to claim 65, wherein the stoppered container comprises an inert stopper.

67. The process according to claim 64, further comprising adjusting the pH of the pamidronate solution to about 5 to about 8.

68. The process according to claim 67, wherein the pH of the pamidronate solution is adjusted to approximately 6.5.

69. A process for manufacturing a solution of pamidronate comprising adding sodium hydroxide solution to pamidronic acid to form the disodium salt of pamidronate.

70. The process according to claim 69, wherein the molar ratio of sodium hydroxide to pamidronic acid is 2:1.

71. The process according to claim 70, wherein additional sodium hydroxide solution is not added to the solution of pamidronate.

72. The solution according to claim 34, further comprising a tonicity adjustment agent.

73. The process according to claim 47, further comprising passing the pamidronate solution through a sterilizing filter before filling the solution into non-reactive plastic containers.

74. An injectable, sterile, ready to use, pyrogen-free pamidronate solution comprising a physiologically accepted alkaline salt of pamidronate which is water soluble and a physiologically acceptable aqueous solvent, having a concentration of between 0.1 and 100 mg/mL, wherein the solution is provided in a sealed non-reactive plastic container.
